(19) United States
(12) Patent Application Publication
(10) Pub. No.: US 2025/0205201 A1
(43) Pub. Date: Jun. 26, 2025

(54) USE OF L-ERGOTHIONEINE FOR ALLEVIATING AND PREVENTING AGE-RELATED LENS DEGENERATION

(71) Applicant: NANJING NUTRABUILDING BIO-TECH CO., LTD., Nanjing (CN)

(72) Inventors: Ronghua YI, Nanjing (CN); Shawn WELLS, Lewisville, TX (US); Kylin LIAO, Nanjing (CN)

(21) Appl. No.: 19/000,869

(22) Filed: Dec. 24, 2024

(30) Foreign Application Priority Data
Dec. 26, 2023 (WO) PCT/CN2023/141751

(51) Int. Cl. A61K 31/4172; A61P 27/02

(52) U.S. Cl. CPC A61K 31/4172; A61P 27/02

(57) ABSTRACT

The present invention provides methods, compositions (e.g., cycdrop or spongarion composition or food or supplement), and uses for alleviating and preventing age-associated lens degeneration in a subject, comprising administrating to the subject in need thereof an effective amount of ergothioneine, or a physiologically acceptable salt, acid, ester, polymer, analog or derivative thereof.

### CROSS REFERENCE TO RELATED APPLICATION

This application claims the priority to a PCT International Application Number PCT/CN2023/141751, filed on Dec. 26, 2023, the content of which is hereby incorporated by reference in its entirety for all purposes.

### FIELD OF THE INVENTION

This invention generally relates to the field of methods for alleviating and preventing age-related (age-associated) lens degeneration in a subject, and more specifically relates to methods comprising administrating to a subject in need thereof: ergothioneine, or a physiologically acceptable salt, acid, ester, polymer, or derivative thereof.

### BACKGROUND

As the human society ages globally, the senescence and degeneration of lens is becoming increasingly common. This phenomenon could be called age-related lens degeneration. As is known to all, if age-related lens degeneration occurs, the eye lens epithelia might turn into opacification, and even ultimately cause blindness, which might bring inconvenience to individuals and increase social burden. Therefore, age-related lens degeneration not only decreases the older adults' quality of life, because of the restricted autonomy, mobility and activities of living, but also has negative social and psychological effects.

There are many reasons contributing to age-related lens degeneration. It is well known that aging is the foremost cause of opacification of the eye lens epithelia, either as a direct result of numerous environmental (trauma, radiation exposure, etc.), nutritional, metabolic injuries or genetic susceptibility or as an indirect result of systemic or ocular diseases including diabetes, glaucoma, and retinal degenerative diseases.

Although surgery to treat age-related lens degeneration, such as management of optimal refractive and glasses for glare reduction, has been considered very successful, surgery-associated complications containing cystoid macular edema and posterior capsular opacification may arise unavoidably and cause irreversible blindness. Thus, it is imperative to identify strategies and antioxidants that would prevent formation of age-related lens degeneration based on the association between age-related lens degeneration and OS. Many strategies and antioxidants in the market seem to attenuate and prevent age-related lens degeneration to the small extend and are not satisfactory now. Therefore, finding improvements in mechanisms urgently to alleviate and prevent opacity of lens with increasing age, is the focus of numerous research programs.

Fortunately, ergothioneine, an efficient and promising substance, has been shown to exhibit powerful antioxidant properties. In the first place, ergothioneine, a common amino acid, occurs naturally in the fungus (mainly in mushrooms) and animal kingdoms (i.e. king crab, etc.). And mammals cannot synthesize ergothioneine in the body directly they can derive it solely through their diet. In addition, ergothioneine is concentrated in cells and tissues wherein exposed to OS frequently, such as ocular tissues (i.e. lens, etc.), liver, bone marrow and seminal fluid. Moreover, Ergothioncine (C9H15N3O2S) is tautomeric and has unique sulfur-based chemistry, making it have an extraordinarily antioxidant potency via many pathways. In a nutshell, ergothioneine could be used as a potential candidate for attenuating age-related lens degeneration by reducing intracellular ROS production and protecting lens epithelial cells (LECs) from oxidative damage.

### SUMMARY

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

One aspect of this invention relates to a method for alleviating and preventing age-associated lens degeneration in a subject, comprising administrating to the subject a composition comprising: an effective amount of ergothioneine, or a physiologically acceptable salt, acid, ester, polymer, or derivative thereof.

In some embodiments, the age-related lens degeneration includes phenomenon comprising hypopsia, visual deterioration, visual disturbance, increasing myopia and blurred vision, opacity of the lens.

In some embodiments, the administration of ergothioneine is capable of scavenging chronic reactive oxygen species to protect cells from oxidative damage, or activating Nrf2-mediated antioxidant genes to maintain the redox balance and protecting lens from oxidation.

In some embodiments, age-related lens degeneration is caused by chronic reactive oxygen species producing in the lens epithelial cells with aging, or caused by decreasing the basic level of Nrf2 with aging.

In some embodiments, the ergothioneine is L-ergothionecine.

In some embodiments, the subject is a mammal. In some embodiments, the subject is an old mammal. In some embodiments, the mammal is a human or an animal (e.g., pet or cattle). For instance, the mammal is a human is aged 30 years or older (e.g., 40 years, 60 years, or 70 years).

In some embodiments, the composition is prepared as a food, a drink, a supplement, a biochemical composition, a care product, pet food, or a nutraceutical composition. In some further embodiments, the composition is administrated in the form of eyedrops or spongarion.

In some embodiments, the administration is through various routes selected from oral, intravenous injection, intramuscular injection, intraperitoneal injection, external use, topical use or sublingual application.

In some embodiments, the composition is formulated in solutions, aqueous suspensions, liquid suspensions, parenteral solutions, injections, microemulsion, (micro) capsules, drops, granules, liquids, powders, aerosols, tonics, syrups, tablets, pills, film, functionalized foods, beverages, toothpaste, nourishments, snacks, gums, bars, sugars, and sublingual articles.

In some embodiments, the ergothioneine is administrated with a daily dose ranging from 2 to 2000 mg. The daily dose may be administrated by a single dose or multiple divided doses. In some embodiments, ergothioneine is administrated at a daily dose of 2-1500 mg, 5-1500 mg, 10-1500 mg, 20-1000 mg, 10-800 mg, 20-600 mg, 30-300 mg, 40-200 mg, 40-100 mg. In some embodiments, ergothioneine is administrated in an amount of 0.1 μM to 1 M or 0.01 to 50.0 wt % (w/w). In some embodiments, ergothioneine, or a physiologically acceptable salt, acid, ester, polymer, or derivative thereof may be administrated in an amount of 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM. In some embodiments, ergothioneine, or a physiologically acceptable salt, acid, ester, polymer, or derivative thereof may be administrated in an amount of 0.05% to 45%, 0.05% to 40%, 0.05% to 30%, 0.05% to 25%, 0.1% to 40%, 0.1% to 30%, 0.1% to 25%, 0.1% to 15%, 0. 5% to 30%, 0.5% to 20%, 0.5% to 10%, 1% to 35%, 1% to 25%, 1% to 10%, 2% to 25%, 2% to 10%, 5% to 9%, 1% to 7%, 2% to 7%, 0.05% to 7%, 0.05% to 9 wt % (w/w).

In some embodiments, ergothioneine is administrated at least once or multiple times a day. In some embodiments, the ergothioneine is administrated daily for at least seven days in one period.

Another aspect of the present invention provides a composition for alleviating and preventing age-associated lens degeneration in a subject, comprising an effective amount of ergothioneine, or a physiologically acceptable salt, acid, ester, polymer, or derivative thereof.

In some embodiments, the ergothioneine is administrated with a daily dose ranging from 2 to 2000 mg. In some embodiments, the ergothioneine is L-ergothioneine.

In some embodiments, the age-related lens degeneration includes phenomenon comprising hypopsia, visual deterioration, visual disturbance, increasing myopia and blurred vision, opacity of the lens.

In some embodiments, the administration of ergothioneine is capable of scavenging chronic reactive oxygen species to protect cells from oxidative damage, or activating Nrf2-mediated antioxidant genes to maintain the redox balance and protecting lens from oxidation.

In some embodiments, the alleviating and preventing of age-associated lens degeneration is ameliorating or alleviating the opacity of lens.

In some embodiments, the administration of ergothioneine is capable of scavenging chronic reactive oxygen species in lens cells to protect cells from oxidative damage, or activating Nrf2-mediated antioxidant genes in lens cells to maintain the redox balance and protecting lens from oxidation.

In some embodiments, the level of chronic reactive oxygen species (ROS) in lens epithelial cells (LEC) decreased by about 20%, 25%, 30%, 35%, 40%, 50%, 55%, 60%, 65%, 70% when administering ergothionine compared with no administration. In some embodiments, the expression of Nrf2 increased by about 40%, 50%, 60%, 80%, 100%, 120%, 140%, 160%, 170%, 180%, 190% when administering ergothionine compared with no administration.

In a further aspect, the present invention provides a use of ergothioneine in a preparation of a composition for alleviating and preventing age-associated lens degeneration in a subject, wherein the composition comprises an effective amount of ergothioncine, or a physiologically acceptable salt, acid, ester, polymer, or derivative thereof.

In some embodiments, the composition is formulated in solutions, aqueous suspensions, liquid suspensions, parenteral solutions, injections, microemulsion, (micro) capsules, drops, granules, liquids, powders, aerosols, tonics, syrups, tablets, pills, film, functionalized foods, beverages, toothpaste, nourishments, snacks, gums, bars, sugars, and sublingual articles. For instance, the composition may be an eyedrop or spongarion.

In some embodiments, the composition comprises ergothioneine with a dose ranging from 2 to 2000 mg. In some embodiments, the ergothioneine is L-ergothioneine.

In some embodiments, the ergothioneine in the composition is capable of scavenging chronic reactive oxygen species to protect cells from oxidative damage, or activating Nrf2-mediated antioxidant genes to maintain the redox balance and protecting lens from oxidation.

In some embodiments, the age-related lens degeneration includes phenomenon comprising hypopsia, visual deterioration, visual disturbance, increasing myopia and blurred vision, opacity of the lens.

### BRIEF DESCRIPTION OF DRAWINGS

FIG. 1 exhibits ROS production in the rats' lenses in groups 1-3.

FIG. 2 is a graph of Nrf2 expression in lens in groups 1-3.

### DETAILED DESCRIPTION

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “effective amount” refers to the amount required to achieve the effect as taught herein. The specific effective dose level for any particular subject will depend upon a variety of factors including the conditions being treated and the severity of the conditions; the specific composition employed; the age, body weight, general health, sex and diet of the subject; the time of administration, route of administration, and rate of excretion of ergothioneine, or a physiologically acceptable salt, polymer, ester, acid, or an analog or derivative employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “physiologically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), in external use, and the like.

In this invention, the inventors have found that ergothioneine can mitigate and prevent age-related lens degeneration effectually and essentially by scavenging intracellular chronic ROS and activating Nrf2-mediated antioxidant genes. Notably, it is the first time to propose a promising ingredient called ergothioneine to attenuate and prevent age-related lens degeneration according to our knowledge.

Various embodiments of the present invention provide for methods for administrating ergothioneine (or an analog or derivative thereof, or a physiologically acceptable salt, acid, ester, polymer, analog or derivative thereof) to a subject for effectively alleviating and preventing age-associated lens degeneration in a subject. For instance, the age-related lens degeneration includes phenomenon comprising hypopsia, visual deterioration, visual disturbance, increasing myopia and blurred vision, opacity of the lens. According to this invention, the administration of ergothioneine is capable of scavenging chronic reactive oxygen species to protect cells from oxidative damage, or activating Nrf2-mediated antioxidant genes to maintain the redox balance and protecting lens from oxidation. Ergothioneine may be administrated in a variety of forms, such as solutions, aqueous suspensions, liquid suspensions, parenteral solutions, injections, microemulsion, (micro) capsules, drops, granules, liquids, powders, aerosols, tonics, syrups, tablets, pills, film, functionalized foods, beverages, toothpaste, nourishments, snacks, gums, bars, sugars, and sublingual articles. For instance, ergothioneine may be administrated (e.g., by oral) with a daily dose ranging from 2 to 2000 mg for at least seven days in one period. Particularly, ergothioneine may be administrated in the form of eyedrops or spongarion. Additionally, the present invention provides a composition for alleviating and preventing age-associated lens degeneration in a subject, comprising an effective amount of ergothioneine, or a physiologically acceptable salt, acid, ester, polymer, or derivative thereof. Further, the present invention also provides use of ergothioneine in a preparation of a composition for alleviating and preventing age-associated lens degeneration in a subject, wherein the composition comprises an effective amount of ergothioneine, or a physiologically acceptable salt, acid, ester, polymer, or derivative thereof.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### EXAMPLES

Ten-day-old suckling rats (Sprague-Dawley, SD) were maintained under the barrier conditions in a temperature-controlled environment and fed a commercial mouse chow. All rat pups received a single subcutaneous injection of sodium selenite (20 μmol/kg body weight) on postpartum day 11. The opacity distribution (by surface plot and plot profile) of the rats' lenses were determined from the captured images by ImageJ analysis software after feeding 7 days. Testing opacity distribution aimed to show whether the formation of age-related lens degeneration is successful. The rats were then injected of sodium selenite every two weeks. And then these induced successfully rats were fed a commercial mouse chow for 15 months if succeeding.

Then these induced successfully rats were randomly divided into three groups: (1) non-supplement group (Group 1, Con, n=10); (2) L-ergothioncine supplement group with low dosage (Group 2, L-ergothioneine, 2 mg/kg/d, n=10); (3) L-ergothioncine supplement group with high dosage (Group 3, L-ergothioneine, 10 mg/kg/d, n=10). All rats were administered L-ergothioneine for once daily by gavage in normal saline solution for 6 months. The opacity distribution (by surface plot and plot profile) of the rats' lenses were observed from the captured images by ImageJ analysis software at the end of this study. The results show that the administration of ergothionine can effectively ameliorate or alleviate the opacity of lens.

At the end of this study, all rats were euthanized by CO₂ inhalation, the eyes were enucleated and the lenses were isolated while viewing the eye under a microscope. Both lenses of each individual rat were processed together to constitute a single value. The isolated lenses of 3 rats in each group were cultured in 2 mL of modified medium-199 with penicillin (100 units/mL), and streptomycin (100 units/mL) for 24 or 48 h at 37° C. under 4% atmospheric oxygen. The LECs from isolated lenses of another 3 rats in each group were cultured overnight in 25 mM glucose-Dulbecco's Modified Eagle's Medium (DMEM).

### Scavenging Intracellular Chronic ROS to Protect Cells from Oxidative Damages

The rat lenses were stained by adding 1 μM of 2′,7′-dichlorodihydrofluorescein diacetate (H2-DCFH-DA) in phosphate buffered saline (PBS) for 30 min at 20° C., then the cells were washed twice with PBS and examined under a fluorescent microscopic with a green filter (450-490 nm) to determine the cytosolic ROS level.

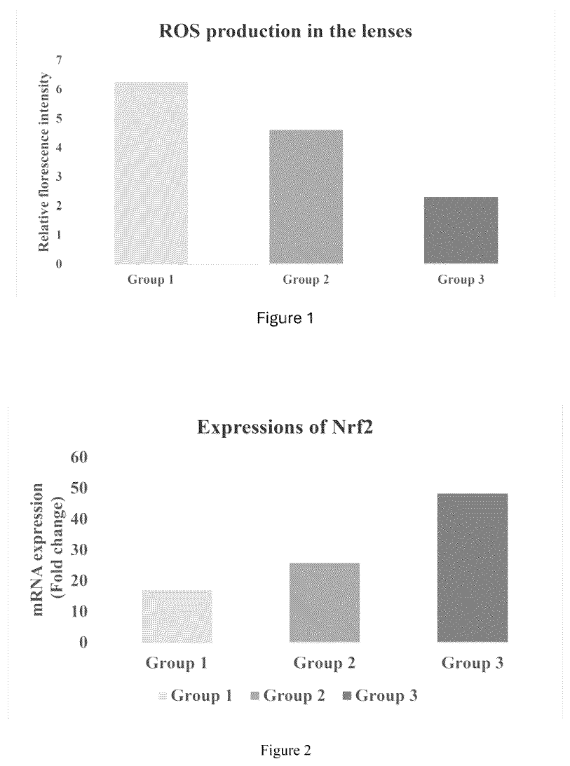

FIG. 1 exhibits ROS production in the rats' lenses in groups 1-3. As illustrated in FIG. 1, supplementation with ergothioneine significantly reduced intracellular chronic reactive oxygen species (ROS) levels in lens epithelial cells (LEC) compared to the non-supplementation group, by more than about 25%. Notably, the high-dose group demonstrated approximately double the efficacy in ROS scavenging compared to the low-dose group.

### Activating Nrf2-Mediated Antioxidant Genes to Maintain the Redox Balance and Protect Lenses from Oxidation

Nrf2 dependent antioxidant protection system plays a crucial role in scavenging ROS generated during various biochemical reactions. Nrf2 is a key transcription factor, which can activate genes regulated by antioxidant response element (ARE), thus up-regulating the expression of a series of antioxidant enzymes, such as superoxide dismutase (SOD), glutathione peroxidase (GSH-Px) and catalase (CAT). These enzymes can effectively eliminate ROS in cells and reduce the damage of oxidative stress to lens cells. Total RNA was extracted from LECs using Trizol reagent. Real-time PCR was measured and performed with the following primers: Nrf2 forward 5′-CCGCCCTCCATATGATGGACTTGGA-3′. RNA samples were reverse-transcribed to cDNA and subjected to quantitative PCR, performed with a Light Cycler 96 Real-Time PCR System using the AceQ qPCR SYBR Green Master Mix. Nrf2 measurements can be used to identify the capacity of ergothioneine-mediated Nrf2.

FIG. 2 is a graph of Nrf2 expression in lens in groups 1-3. As shown in FIG. 2, the inventors have found increased mRNA level of Nrf2 in LECs. The low-dose group exhibited an approximate 50% improvement compared to placebo, while the high-dose group demonstrated a remarkable enhancement of around 180% over placebo.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method for alleviating and preventing age-associated lens degeneration in a subject, comprising administrating to the subject a composition comprising: an effective amount of ergothioneine, or a physiologically acceptable salt, acid, ester, polymer, or derivative thereof.

2. The method of claim 1, wherein the age-related lens degeneration includes phenomenon comprising hypopsia, visual deterioration, visual disturbance, increasing myopia and blurred vision, opacity of the lens.

3. The method of claim 1, wherein the administration of ergothioneine is capable of scavenging chronic reactive oxygen species to protect cells from oxidative damage, or activating Nrf2-mediated antioxidant genes to maintain the redox balance and protecting lens from oxidation.

4. The method of claim 1, wherein the alleviating and preventing age-associated lens degeneration is ameliorating or alleviating the opacity of lens.

5. The method of claim 1, wherein the administration of ergothioneine is capable of scavenging chronic reactive oxygen species in lens cells to protect cells from oxidative damage, or activating Nrf2-mediated antioxidant genes in lens cells to maintain the redox balance and protecting lens from oxidation.

6. The method of claim 1, wherein the level of chronic reactive oxygen species (ROS) in lens epithelial cells (LEC) decreased by about 20%, 25%, 30%, 35%, 40%, 50%, 55%, 60%, 65%, 70% when administering ergothionine compared with no administration.

7. The method of claim 1, wherein the expression of Nrf2 increased by about 40%, 50%, 60%, 80%, 100%, 120%, 140%, 160%, 170%, 180%, 190% when administering ergothionine compared with no administration.

8. The method of claim 1, wherein the ergothioneine is L-ergothioneine.

9. The method of claim 1, wherein the subject is a mammal.

10. The method of claim 1, wherein the composition is prepared as a food, a drink, a supplement, a biochemical composition, a care product, pet food, or a nutraceutical composition.

11. The method of claim 1, wherein the composition is administrated in the form of eyedrops or spongarion.

12. The method of claim 1, wherein the administration is through various routes selected from oral, intravenous injection, intramuscular injection, intraperitoneal injection, external use, topical use or sublingual application.

13. The method of claim 1, wherein the composition is formulated in solutions, aqueous suspensions, liquid suspensions, parenteral solutions, injections, microemulsion, (micro) capsules, drops, granules, liquids, powders, aerosols, tonics, syrups, tablets, pills, film, functionalized foods, beverages, toothpaste, nourishments, snacks, gums, bars, sugars, and sublingual articles.

14. The method of claim 1, wherein the ergothioneine is administrated with a daily dose ranging from 2 to 2000 mg.

15. The method of claim 1, wherein the ergothioneine is administrated in an amount of 0.1 μM to 1 M or 0.01 to 50.0 wt % (w/w).
